(19) United States
(12) Patent Application Publication
(10) Pub. No.: US 2023/0251180 A1
(43) Pub. Date: Aug. 10, 2023

(54) MEASURING DEFORMABILITY OF A CELL VIA A PRESSURE FIELD

(71) Applicant: Hewlett-Packard Development Company, L.P., Spring, TX (US)

(72) Inventors: Fausto D'Apuzzo, Palo Alto, CA (US); Viktor Shkolnikov, Palo Alto, CA (US); Alexander N. Govyadinov, Corvallis, OR (US)

(73) Assignee: Hewlett-Packard Development Company, L.P., Spring, TX (US)

(21) Appl. No.: 18/012,632

(22) PCT Filed: Jul. 8, 2020
(86) PCT No.: PCT/US2020/041232

§ 371 Date: Dec. 22, 2022

(51) Int. Cl. G01N 15/14; B01L 3/00

(52) U.S. Cl. CPC G01N 15/1404; G01N 15/1434; B01L 3/502761; G01N 2015/1495; B01L 2300/0654; B01L 2200/0663; B01L 2400/086; B01L 2400/0487

(57) ABSTRACT

An example method for measuring deformability of a cell via a pressure field, consistent with the present disclosure, includes flowing a biologic sample containing a plurality of cells along a first fluidic channel and into an intersection between the first fluidic channel and a second fluidic channel of a microfluidic device. The method includes introducing a pressure field at the intersection and into the first fluidic channel via the second fluidic channel and a plurality of apertures in a channel wall disposed in the intersection. The method further includes measuring deformability of a cell among the plurality of cells responsive to the introduction of the pressure field.

### BACKGROUND

Cellular mechanical properties may be indicative of various disease states. For example, a change in the deformability of red blood cells is an early indication of sepsis, as well as hereditary disorders, such as spherocytosis, elliptocytosis, ovalocytosis, and stomatocytosis, metabolic disorders, such as diabetes, hypercholesterolemia, and obesity, as well as other disorders, such as adenosine triphosphate-induced membrane changes, oxidative stress, and paroxysmal nocturnal hemoglobinuria. A change in red blood cell deformability may be associated with malaria, sickle cell anemia, and myocardial infarction. As a further example, a change in deformability of white blood cells may also be be associated with sepsis.

Rheological phenotyping, or the characterization of the deformability of cells, allows for detection of various diseases. In cancer research, elasticity of circulating tumor cells is strongly correlated to the metastatic potential of the cells, with more elastic cells having higher metastatic potential.

### BRIEF DESCRIPTION OF THE DRAWINGS

FIG. 1 illustrates an example method for measuring deformability of a cell, consistent with the present disclosure.

FIG. 2 illustrates an example apparatus for measuring deformability of a cell, consistent with the present disclosure.

FIG. 3 illustrates another example apparatus for measuring deformability of a cell, consistent with the present disclosure.

FIG. 4 illustrates another example apparatus for measuring deformability of a cell, consistent with the present disclosure.

FIG. 5 illustrates another example apparatus for measuring deformability of a cell, consistent with examples of the present disclosure.

FIG. 6 illustrates another example apparatus for measuring deformability of a cell, consistent with examples of the present disclosure.

FIGS. 7A-7B illustrate an example apparatus for measuring deformability of a cell including a barrier to contain the cell, consistent with examples of the present disclosure.

FIG. 8 illustrates another example apparatus for measuring deformability of a cell, consistent with examples of the present disclosure.

FIG. 9 illustrates another example apparatus for measuring deformability of a cell, consistent with the present disclosure.

FIG. 10 illustrates another example apparatus for measuring deformability of a cell, consistent with the present disclosure.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized, and structural or logical changes may be made without departing from the scope of the present disclosure. The following detailed description, therefore, is not to be taken in a limiting sense, and the scope of the present disclosure is defined by the appended claims. It is to be understood that features of the various examples described herein may be combined, in part or whole, with each other, unless specifically noted otherwise.

Biological cells are the basic building blocks of skin, tissues, and other materials. Cells and their organelles are enveloped by thin membranes that separate their chemical contents from the extracellular environment. Accurate measurement of cell mechanical deformability properties, such as on the single cell level, is useful for basic biological research, as well as for diagnostic purposes and for isolation of particular cell populations of interest. In addition, basic biological studies and clinical applications seek to determine both the full non-linear stress-strain response of the cell and the visco-elastic properties (e.g., complex elastic moduli) of the cell. The stress-strain response and the visco-elastic properties assist in predicting the cell migration through tissues, the cell potential for wound healing (e.g., tissue reconfiguration), the metastatic of the cell potential, and other biomechanical cell behaviors.

Cell deformation and mechanical property analysis may allow for rheological phenotyping. For instance, cells may be input into an apparatus and deformed. The flow may be driven by pumps, and the deformation of the cell may be observed using a high-speed camera. The deformation is obtained by post-processing. Post-processing a large number of optical images, such as for processing large amounts of cells (e.g., greater than 10\hat{ }6 cells), takes a significant amount of time and is poorly amenable to point-of-care instrument solutions. Moreover, post-processing deformation information may reduce the ability to sort the cells, as the information is not processed in real time.

Accordingly, disclosed herein is a compact, integrated microfluidic apparatus compatible with integration into point-of-care instruments to perform mechanical cell deformation analysis and, optionally, cell sorting. The apparatus includes two intersecting fluidic channels which are actuated in crossing directions by fluidic pumps to flow a biologic sample in a first direction and to provide a constant flow of fluid, sometimes herein referred to as a “pressure field”, at a channel wall at the intersection of the two fluidic channels. The channel wall includes a plurality of apertures which create a structured pressure field by segmenting the constant flow of fluid into different pressure zones that are applied to a cell to be analyzed at the intersection. By applying a structured pressure field to the cell, an imaging system may be used that operates at relatively low speed. Such low-speed imaging systems may capture approximately ten to one-hundred frames per second (fps), as compared to relatively higher speed imaging systems, such as those that capture approximately 1000 fps or more. The use of slower fps imaging systems may reduce the cost for conducting rheological phenotyping, and analytic rheological phenotyping may be performed on a single cell or a few cells at a time. Furthermore, manufacturing costs may be reduced as the structured pressure field is generated using simple fluid pumps, which may be on-device or off-device, as compared to use of high-frequency actuators (e.g., piezo inkjet (PIJs)) and/or ultrasound sources.

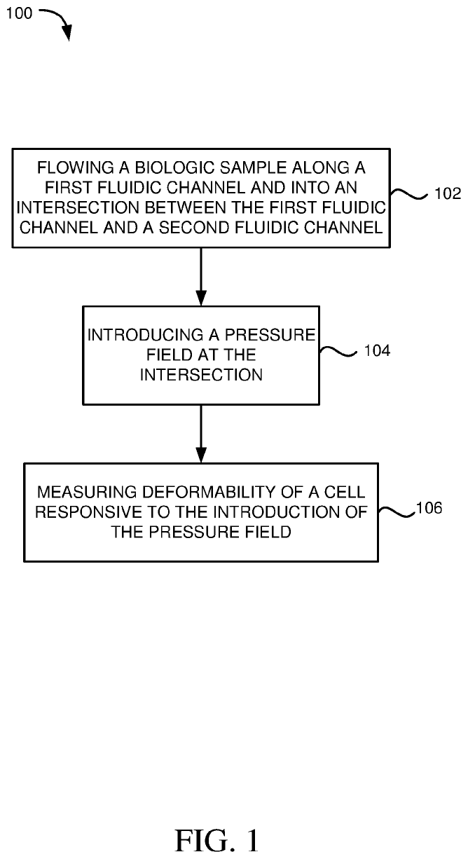

Turning now to the figures, FIG. 1 illustrates an example method for measuring deformability of a cell, consistent with the present disclosure. At 102, the method 100 includes flowing a biologic sample containing a plurality of cells along a first fluidic channel and into an intersection between the first fluidic channel and a second fluidic channel of a microfluidic device. As further described herein, flow within the microfluidic device may be controlled using a set of fluidic pumps, and an imaging system may be used to visualize cells within the microfluidic device. For example, the microfluidic device includes the first fluidic channel and the second fluidic channel that are actuated in crossing directions by the set of pumps, such as on-device or off-device fluidic pumps. In various examples, the microfluidic device may include a cross-channel which is actuated in two directions by the set of pumps.

At 104, the method 100 further includes introducing a pressure field at the intersection and into the first fluidic channel via the second fluidic channel and a plurality of apertures in a channel wall disposed in the intersection. Introducing the pressure field may include generating a cross-directional flow to the flow of the biologic sample through the first fluidic channel. The cross-directional flow may be a constant flow of fluid that is driven along the second fluidic channel toward the first channel wall. In some examples, introducing the pressure field includes driving a flow of fluid along the second fluidic channel and toward the channel wall via the pressure field, and segmenting the flow of the fluid into different pressures zones in the intersection via the plurality of apertures in the channel wall. The plurality of apertures may segment the constant flow of fluid into the different pressure zones within the intersection, thereby creating a structured pressure field that interacts with the cell. The different pressure zones may include relatively-high pressure zones and relatively-low pressure zones that provide local gradients of pressure to probe the cell deformability. Accordingly, as used herein, a structured pressure field includes or refers to a pressure field having different pressure zones.

In some examples, once a cell is identified within the intersection, the first fluidic pump may stop pumping and therefore stop the flow of fluid through the microfluidic device. The intersection between the fluidic channels may include a cell probing chamber, which is arranged with an imaging system to visualize the cells. In specific examples, the method 100 further includes isolating the cell in the cell probing chamber of the microfluidic chip by terminating the flow of the biologic sample through the microfluidic chip. For example, the method 100 may further include detecting the cell of the biologic sample in the cell probing chamber, and isolating the cell in the intersection by reducing the flow of the biologic sample through the first fluidic channel. The cell may be sensed using a sensor disposed proximate to the intersection. In such examples, the method 100 may further include sensing entry of the cell into the intersection via a signal received from the sensor disposed proximate to the intersection. Example sensors may include impedance sensors, capacitance sensors, optical sensors, thermal sensors, voltammetric sensors, amperometric/coulometric sensors, and transistors, among other types of sensors capable of detecting presence of a cell. As a specific example, flowing the cell into the intersection may include flowing the biologic sample in a first direction along the first fluidic channel via a first fluidic pump, reducing the flow in the first direction in response to the cell being located in the intersection, and introducing the pressure field in a second direction that crosses the first direction via a second fluidic pump.

At 106, the method 100 includes measuring deformability of the cell among the plurality of cells responsive to the introduction of the pressure field. For instance, the method 100 may include measuring the size and/or shape, such as the width, length, and/or diameter, of the cell before application of the structured pressure field, and measuring the size and/or shape of the cell during application of the structured pressure field to measure the deformability. As discussed further herein, the microfluidic device may include a transparent surface that allows imaging of the intersection, such as by epi-illumination microscopy.

Accordingly, the method 100 may include imaging the cell while the pressure field causes deformation of the cell via the imaging system and measuring deformability of the cell based on image data received from the imaging system. In some examples, a single cell may be imaged and, in other examples, multiple cells may be imaged at one time, such as two cells, three cells or more. In various examples, multiple images of a cell may be obtained. As a specific example, an image of the cell may be captured before, during, and after applying the pressure field. By capturing the image of the cell before, during, and after applying the pressure field, the non-deformed geometry is captured, as well as the early stages of the deformation.

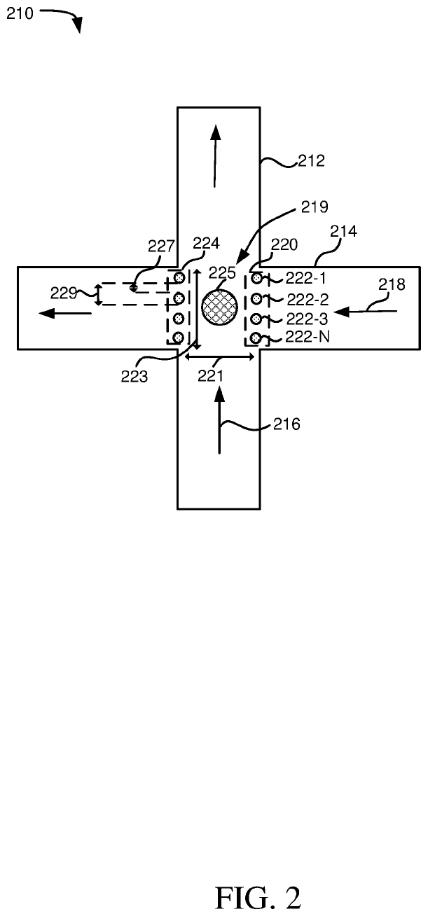

FIG. 2 illustrates an example apparatus for measuring deformability of a cell, consistent with the present disclosure. The apparatus 210 is capable of performing the method 100 illustrated in FIG. 1. For instance, the apparatus 210 may measure deformability of a cell 225 of a biologic sample in an intersection 219 of two fluidic channels 212, 214 of the apparatus 210 via a pressure field.

In specific examples, the apparatus 210 is implemented as a microfluidic device. The apparatus 210 includes a first fluidic channel 212 and a second fluidic channel 214 that intersects the first fluidic channel 212. The first fluidic channel 212 and the second fluidic channel 214 are actuated in crossing directions by a set of fluidic pumps. The microfluidic device may include the set of fluidic pumps and in other examples, the set of fluidic pumps are off-device and are coupled to the microfluidic device. In any example, the set of fluidic pumps include a first fluidic pump coupled to the first fluidic channel 212 to flow a biologic sample in the first direction 216 of the crossing directions, and a second fluidic pump coupled to the second fluidic channel 214 to flow fluid in a second direction 218 of the crossing directions. Example pumps include an integrated inertial pump, a thermal inkjet (TIJ) resistor, a piezoelectric device, a magnetostrictive element, an ultrasound source, and other suitable pumps.

The apparatus 210 further includes a first channel wall 220 disposed at the intersection 219 of the first fluidic channel 212 and the second fluidic channel 214. The first channel wall 220 includes a plurality of apertures that provide fluidic communication between the first fluidic channel 212 and the second fluidic channel 214. In the specific example illustrated by FIG. 2, the first channel wall 220 includes a plurality of pillar structures 222-1, 222-2, 222-3, 222-N that are spaced periodically in the intersection 219 to form the plurality of apertures. The pillar structures 222-1, 222-2, 222-3, 222-N are not limited to that illustrated by FIG. 2 and may comprise a variety of geometric shapes, such as elongated pillars and/or different geometric shaped pillars. In other examples, the first channel wall 220 may include a solid structure with apertures formed therein, with the apertures providing fluidic communication between the second fluidic channel 214 and the intersection 219.

Fluid, including a biologic sample, may be input at a fluidic input coupled to the first fluidic channel 212, may be flowed through the apparatus 210 in the first direction 216, and may exit the apparatus 210 at a fluidic output. As noted above, the flow of fluid is controlled by fluidic pumps. For instance, the fluidic pumps (not illustrated in FIG. 2) may be disposed within the first fluidic channel 212 and within the second fluidic channel 214. The flow within the apparatus 210 may be controlled by individually actuating the different fluidic pumps. To induce flow from the fluidic input to the fluidic output in the first direction 216, a fluidic pump near the fluidic input may be actuated. To reverse the flow and/or slow the flow of fluid from fluidic input to fluidic output, the fluidic pump near the fluidic input may cease firing or fire at a slower rate, and/or a fluidic pump near the fluidic output may be actuated. To stop the flow of fluid, all fluidic pumps may cease firing. Similarly, to induce flow along the second fluidic channel 214 in the second direction 218 and toward the intersection 219, a fluidic pump near the arrow 218 may be actuated. In some examples, the fluidic pumps are integrated with the microfluidic device and may be TIJ resistors, among other examples. Additionally and/or alternatively, an external pump or external pumps may be used to induce a fluid flow in the microfluidic device.

The cell 225 may be isolated in the intersection 219. In various examples, the intersection 219 forms a cell probing chamber of the microfluidic device. A cell probing chamber may hold a threshold number of cells, such as a single cell, from a biologic sample for deformation analysis. The threshold number of cells may include five cells, four cells, three cells, two cells, or a single cell, in various examples. In some examples, the intersection 219 with the structured pressure field may have a width 221 and/or a length 223 that exceeds a size of a single target cell to allow for simultaneous deformation interrogation and/or analysis of multiple cells in the intersection 219 at the same time. As a specific example, the intersection 219 may have a width 221 of a size sufficient to allow one cell to fit, which may mitigate or prevent side-by-side cell arrangement along the width 221, and the intersection 219 may have a length 223 that significantly exceeds the cell size, and which may allow for multiple cells to be arranged or lined up one-by-one along the length 223 of the intersection 219 at a given time. In various examples, the cell 225 is isolated in the intersection 219 by terminating or reducing the flow of the biologic sample through the microfluidic device. While complete flow termination may be difficult to achieve, significant reduction of the flow rate (or slow down of the flow) may enable enough time for deformation analysis to be performed. Although examples are not so limited and as further described below, fluid may flow within the first fluidic channel 212 and the second fluidic channel 214 in a variety of ways, based on operation of the fluidic pumps. In some examples, deformation analysis may be performed during continuous flow and/or pulsatory flow of the biologic sample through the first fluidic channel 212, and/or continuous flow and/or pulsatory flow of fluid through the second fluidic channel 214.

As discussed above, the apparatus 210 may cause deformation of the cell 225 by introducing a structured pressure field into the intersection 219. The first channel wall 220 may allow for performing deformation analysis on the cell 225 by creating the structured pressure field when fluid flows through the apertures of the first channel wall 220, and the structured pressure field is applied to the cell 225 in the intersection 219. As a specific example, the first fluidic pump generates a first pressure field and flows the biologic sample containing a plurality of cells along the first fluidic channel 212 in the first direction 216 of the crossing directions. The second fluidic pump generates a second pressure field and flows fluid along the second fluidic channel 214 in the second direction 218 of the crossing directions toward the intersection 219 and through the plurality of apertures of the first channel wall 220 responsive to a cell 225 of the plurality of cells being located in the intersection 219. The plurality of apertures of the first channel wall 220 modify the second pressure field to create the structure pressure field. The structured pressure field created by the apertures includes different pressure zones which are provided in the intersection 219 and applied to the cell 225 to cause deformation.

To view the cell 225 and measure deformability, the intersection 219 of the apparatus 210 may include or be coupled to integrated optics and/or an external imaging system. The integrated optics may include lenses, such as micro-lenses packaged with the microfluidic chip, or flat-lenses which are fabricated directly or packaged with the microfluidic capping layer, or imaged through lens-less computational microscopy.

In some examples, the apparatus 210 further includes a second channel wall 224 disposed at the intersection 219. As shown, the second channel wall 224 is opposite the first channel wall 220 within the intersection 219. The second channel wall 224 may be symmetrical to the first channel wall 220, although examples are not so limited. In some examples, the second channel wall 224 includes a plurality of pillars with spacing between them that form a plurality of apertures. Although examples are not so limited and the second channel wall 224 may be a filter formed by a material having apertures therein. In various examples, the second channel wall 224 may be used to filter material from the biologic sample. The material filtered out may include debris and other material of a size that is less than the apertures in the second channel wall 224. In such examples, the second fluidic channel 214 may be coupled to a waste reservoir at a fluidic output of the second fluidic channel 214.

In various examples, the structures of the first channel wall 220 (and optionally the second channel wall 224) provide the different pressure zones in the intersection 219 when fluid flows therethrough based on a width 227 of the apertures and a pitch 229 between the structures of the first channel wall 220. The structures may include the pillars, portions of a material of the wall, and/or elongated pillars or structures, as further illustrated herein. More particularly, the velocity distribution of the fluid may exert a spatially dependent pressure on the walls of the cell(s) in proximity to the first channel wall 220. The width 227 of the apertures may be in the order of 1-10 micrometers (μm), and in some examples, a width 227 of 1-5 μm, and with a pitch 229 in the order of 2-100 μm. In some specific examples, the pitch 229 may be in the order of 2-10 μm or 2-5 μm. Both the width 227 and the pitch 229 may be smaller than the size of the cell of interest, which allows for several jets of fluid in the second direction 218 for a cell 225 while isolated in the intersection 219 (e.g., ten jets). The width 227 and pitch 229 may be variable to optimize deformation interrogation frequencies, flows, and pressure.

The flow of the biologic sample in the first direction 216 may be synchronized with the flow of the fluid in the second direction 218. The synchronization may allow for use of a low speed imaging system to image the deformation of the cell 225. For example, the flow of fluid in the first direction 216 may occur step-wise with the flow of fluid in the second direction 218, thereby synchronizing the flow of the biologic sample along the first fluidic channel 212 with the jetting of fluid along the second fluidic channel 214. As a specific example, the first fluidic pump pulses the first pressure field to flow the biologic sample in the first direction 216 at a first time, and then the second fluidic pump pulses the second pressure field at a second time to create the structured pressure field in the intersection 219 via flow of fluid in the second direction 218. The first fluidic pump pulses a third pressure field to then flow the biologic sample in the first direction 216 at a third time. The second pressure field may be longer in time than the first pressure field and third pressure field, such that multiple image frames of the cell 225 may be obtained. Similarly, the flow of fluid in the second direction 218 may be faster than the flow of fluid in the first direction 216, to allow for use of a low speed imaging system and/or a stroboscopic imaging system. Although examples are not so limited and may include concurrent actuation of the fluidic pumps.

Although FIG. 2 (and various figures herein) illustrate the pillar structures and apertures as having symmetric distribution, examples are not so limited. For example, the pillars and/or apertures may be different shapes and/or have variable widths 227 and pitches 229 between different structures of the first channel wall 220. Such variability may be used with a high-speed imaging system, which may not use synchronization between the flow of fluid in the first direction 216 and in the second direction 218. Additionally, pillars are not limited to a cylinder shape, and may include a variety of geometric shapes, such as various prisms (e.g., triangular prisms, rectangular prisms, pentagonal prism, hexaconal prisms), cones, pyramids, among other shapes.

Further, although FIG. 2 illustrates cross-shaped fluidic channels, examples are not so limited. For instance, the apparatus 210 may include a first fluidic channel which is intersected by a second fluidic channel forming a T-flow array, as further illustrated herein. In other examples, the first fluidic channel may be intersected by a plurality of second fluidic channels. Examples are additionally not limited to the first fluidic channel 212 and the second fluidic channel 214 being orthogonal to one another, and the fluidic channels 212, 214 may be arranged at a variety of angles with respect to one another.

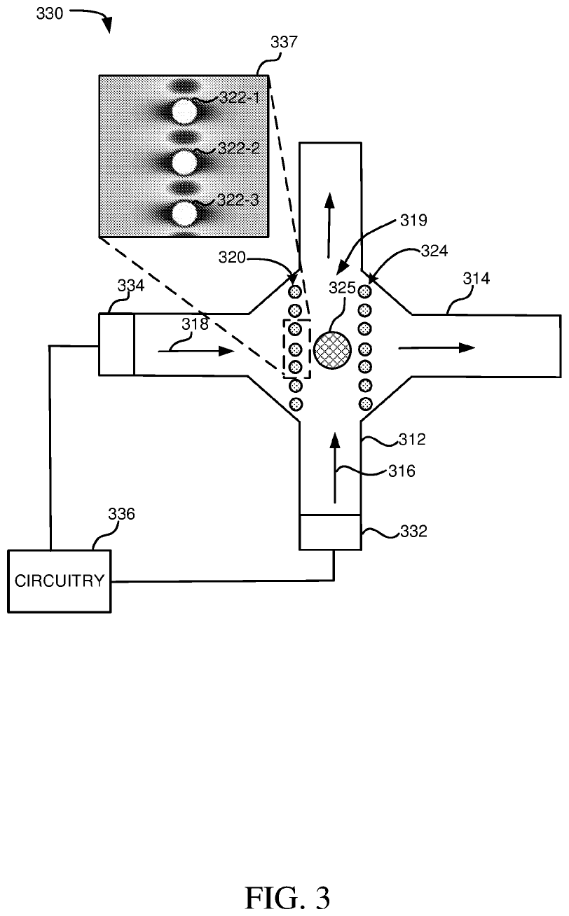

FIG. 3 illustrates another example apparatus for measuring deformability of a cell, consistent with the present disclosure. As with the apparatus 210 of FIG. 2, the apparatus 330 of FIG. 3 includes a microfluidic device having a first fluidic channel 312, a second fluidic channel 314 that intersects the first fluidic channel 312, and a first channel wall 320 disposed at the intersection 319. In some examples, the second fluidic channel 314 is disposed orthogonal to the first fluidic channel 312, although examples are not so limited. The first channel wall 320 includes a plurality of apertures that provide fluidic communication between the first fluidic channel 312 and the second fluidic channel 314. The apparatus 330 further includes a first fluidic pump 332 coupled to the first fluidic channel 312, a second fluidic pump 334 coupled to the second fluidic channel 314, and circuitry 336. The first fluidic pump 332 and second fluidic pump 334 may form part of the microfluidic device or may be separate from the microfluidic device. As previous described, the apparatus 330 may further include a second channel wall 324, although examples are not so limited.

The circuitry 336 may activate the first fluidic pump 332 to generate a first pressure field and to flow a biologic sample containing a plurality of cells, via the first pressure field, along the first fluidic channel 312 in a first direction 316 and toward the intersection 319 of the first fluidic channel 312 and the second fluidic channel 314. The circuitry 336 may activate the second fluidic pump 334 to generate a second pressure field and to flow fluid, via the second pressure field, along the second fluidic channel 314 in a second direction 318 toward the intersection 319 and through the first channel wall 320. As illustrated, the first direction 316 and second direction 318 cross one another.

The activation of the first fluidic pump 332 and second fluidic pump 334 may be at separate times to synchronize the flow of the biologic sample through the intersection 319 with flow of the structured pressure field. Although examples are not so limited, and in some examples, the first fluidic pump 332 and second fluidic pump 334 may be activated at the same time.

The circuitry 336 may further measure deformability of a cell 325 among the plurality of cells responsive to the introduction of the second pressure field. As previously described, the first channel wall 320 may include a plurality of pillars (or obstacles) in the flow path of the second pressure field. The pillars, such as the illustrated pillars 322-1, 322-2, 322-3 in the insert 337, provide a velocity distribution of the fluid which causes different pressure zones by modifying the second pressure field. The insert 337 illustrates an example of the different pressure zones created by the pillars 322-1, 322-2, 322-3. The different pressure zones effectively exert a spatially dependent pressure on the wall(s) of the cell 325 in proximity to the first channel wall 320. While the structured pressure field is exerted on the cell 325, deformation of the cell 325 may be measured, such as by an imaging system. As further described herein, the first fluidic channel 312, the second fluidic channel 314, and the intersection 319 may include a transparent lid disposed over a base substrate to form a cross-channel, which may be imaged by the imaging system.

In accordance with various examples, fluid may flow through example microfluidic devices, such as within the first fluidic channel 312 and the second fluidic channel 314 of the microfluidic device illustrated by FIG. 3, in a variety of ways, based on operation of the internal or external fluidic pumps. In some examples, deformation analysis may be performed during continuous flow of the biologic sample through the first fluidic channel 312 and/or continuous flow of fluid through the second fluidic channel 314. For example, the deformation analysis may be performed without a change in the flow rate of the biologic sample through first channel 312. In such examples, the biologic sample may be diluted and passed through the first fluidic channel 312 such that cells are passed through the intersection 319 a threshold number of cells at a time, such as one-by-one, and imaged via the imaging system at a synchronized rate with a firing rate of the second fluidic pump 334 driving the flow of fluid in the second fluidic channel 314. In specific examples, a continuous flow may be used in both the first fluidic channel 312 and the second fluidic channel 314, and the resulting structured pressure field exerted on the cell 325 in the intersection 319 may be a steady state pressure field.

In various examples, a pulsatory flow may be used to move cells through the first fluidic channel 312 and/or flow fluid through the second fluidic channel 314, rather than terminating or reducing the flow rate(s). For instance, the first fluidic pump 332 may fire periodically and move cells through the intersection 319 along the first fluidic channel 312. When the flow is produced by a pulsating pump such as an inertial pump, the flow rate per pulse of the inertial pump is well defined and cells are transported through the intersection 319 with precision steps. Similarly, the second fluidic pump 334 may fire periodically and move fluid through the intersection 319 along the second fluidic channel 314 to create the structured pressure field to be applied to cells. When a pulsatory flow is used in either or both of the first fluidic channel 312 and the second fluidic channel 314, the structured pressure field introduced in the intersection 319 may not be a steady state field. In examples in which a pulsatory flow is used in the second fluidic channel 314 or both the first fluidic channel 312 and the second fluidic channel 314, the resulting structured pressure field exerted on the cell 325 in the intersection 319 may be a non-stationary interrogating cell pressure field, which may be referred to as synthetic jet.

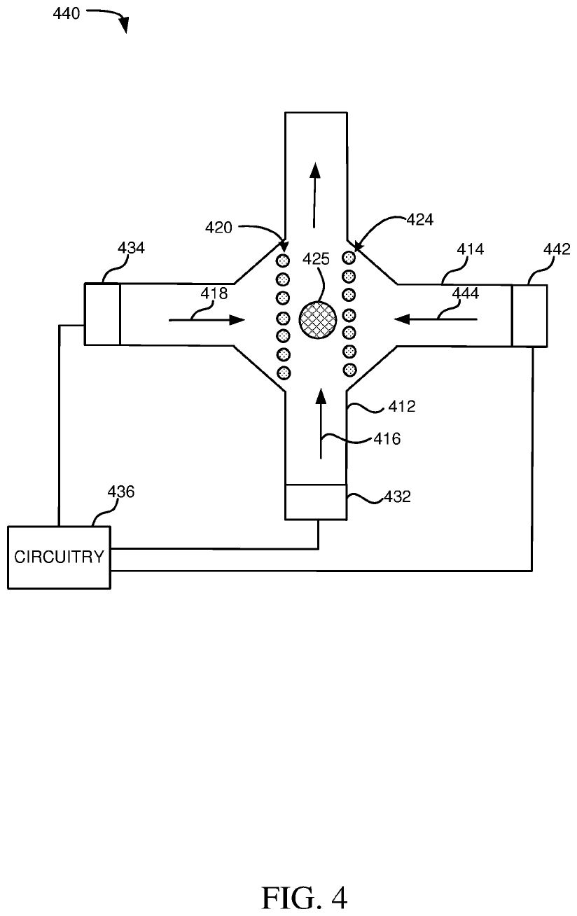

FIG. 4 illustrates another example apparatus for measuring deformability of a cell, consistent with the present disclosure. The apparatus 440 of FIG. 4 is similar to the apparatus 330 of FIG. 3, with the addition of a third fluidic pump 442. The second fluidic pump 434 and third fluidic pump 442 may provide converging pressure fields used to deform a cell 425 located in the intersection of two fluidic channels 412, 414.

The apparatus 440 includes a microfluidic device having a first fluidic channel 412, a second fluidic channel 414 that intersects the first fluidic channel 412, and a first channel wall 420 and a second channel wall 424 disposed at the intersection. The first channel wall 420 and the second channel wall 424 may each include a plurality of apertures that provide fluidic communication between the first fluidic channel 412 and the second fluidic channel 414. The apparatus 440 may further include a first fluidic pump 432 coupled to the first fluidic channel 412, a second fluidic pump 434 coupled to the second fluidic channel 414, the third fluidic pump 442 coupled to the second fluidic channel 414, and circuitry 436. The first fluidic pump 432, second fluidic pump 434, and third fluidic pump 442 may form part of the microfluidic device or may be separate therefrom.

As previously described, the circuitry 436 may activate the first fluidic pump 432 to generate a first pressure field and to flow a biologic sample, via the first pressure field, along the first fluidic channel 412 in the first direction 416, and may activate the second fluidic pump 434 to generate a second pressure field and to flow fluid, via the second pressure field, along the second fluidic channel 414 in the second direction 418 toward the intersection and through the first channel wall 420. The circuitry 436 may further activate the third fluidic pump 442 to generate a third pressure field and to flow fluid, via the third pressure field, along the second fluidic channel 414 in a third direction 444 and through the second channel wall 424. As shown, the second pressure field and the third pressure field converge at the intersection of the fluidic channels 412, 414, and the second direction 418 and the third direction 444 are cross directional to the flow of the biologic sample through the first fluidic channel 412 in the first direction 416.

The second fluidic pump 434 and the third fluidic pump 442 may be activated at the same time, such that the second pressure field and the third pressure field converge at the intersection of the first fluidic channel 412 and the second fluidic channel 414. The activation of the first fluidic pump 432 may be at a separate time from the activation of the second fluidic pump 434 and the third fluidic pump 442 to synchronize the flow of the biologic sample through the intersection along the first fluidic channel 412 with the converging structured pressure fields. Although examples are not so limited, and in some examples, the first fluidic pump 432 may be activated at the same time as the second fluidic pump 434 and the third fluidic pump 442.

The circuitry 436 may measure deformability of a cell 425 responsive to the introduction of the second pressure field and the third pressure field. The use of three fluid flows may provide a structured pressure field via two equal counter flows (e.g., the second pressure field and the third pressure field). The counter flows may define the structured pressure field and the first fluid flow may define a speed of flow of the biologic sample, such as a cell travel speed. Use of the two equal counter flows may prevent or mitigate side shift of the cell 425 under deformation interrogation, and may simplify apparatus control. In other examples and/or in addition, the intersection may include a barrier used to prevent or mitigate side shift, as further illustrated herein.

The flow of fluid within the first fluidic channel 412 and the second fluidic channel 414, via the first fluidic pump 432, the second fluidic pump 434, and the third fluidic pump 442, may be continuous flows and/or pulsatory flows, as described above. As the two flows of fluid in the second fluidic channel 414 are counter flows, the flows may both be continuous flows or both be pulsatory flows, with the flow of the biologic sample in the first fluidic channel 412 being either a continuous flow or a pulsatory flow, in various examples.

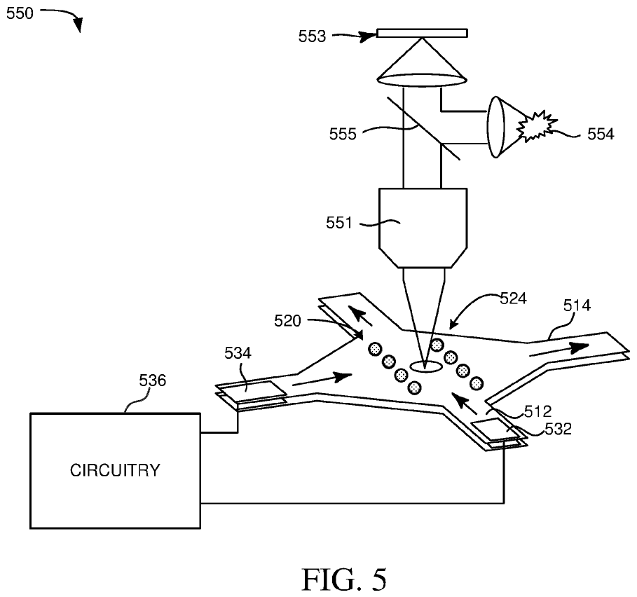

FIG. 5 illustrates another example apparatus for measuring deformability of a cell, consistent with examples of the present disclosure. The apparatus 550 of FIG. 5 is similar to the apparatus 330 of FIG. 3 with the addition of an imaging system coupled to the microfluidic device. As previously described, the apparatus 550 includes a microfluidic device having a first fluidic channel 512 coupled to a first fluidic pump 532, a second fluidic channel 514 coupled to a second fluidic pump 534, a first channel wall 520 at the intersection, and an optional second channel wall 524 at the intersection, and circuitry 536 coupled to the microfluidic device. The circuitry 536 controls actuation of the fluidic pumps 532, 534. For example, the circuitry 536 may coordinate the actuation of the fluidic pumps 532, 534 to create structured pressure fields in the intersection of the two fluidic channels 512, 514. The circuitry 536 may actuate the first fluidic pump 532 to drive a portion of the biologic fluid toward the intersection along the first fluidic channel 512, and actuate the second fluidic pump 534 to drive fluid toward the intersection along the second fluidic channel 514 and through the first channel wall 520 to apply a structured pressure field on a cell within the intersection.

The first fluidic channel 512, the second fluidic channel 514, and/or the intersection may include a transparent lid disposed over a base substrate to form a cross-channel. The intersection may be imaged to measure deformation of a cell due to the structured pressure field applied to the cell.

As illustrated in FIG. 5, the apparatus 550 may include an imaging system arranged with the microfluidic device to capture image data of the intersection. The circuitry 536 may measure the deformability of the cell based on the image data received from the imaging system. The imaging system may include an image sensor 553, which may be a charge-couple device (CCD), a complementary metal-oxide-semiconductor (CMOS) imaging device, or any other suitable imaging sensor. The imaging system may further include a light source 554, a dichroic mirror 555, and an objective 551 (or a lens, such as a flat lens) to visualize the cell located in the intersection of the fluidic channels 512, 514.

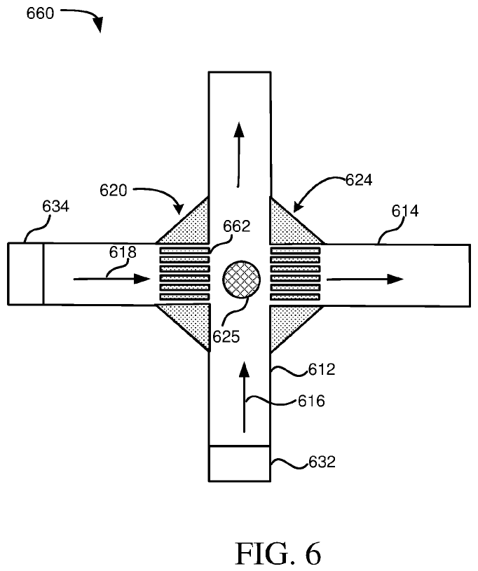

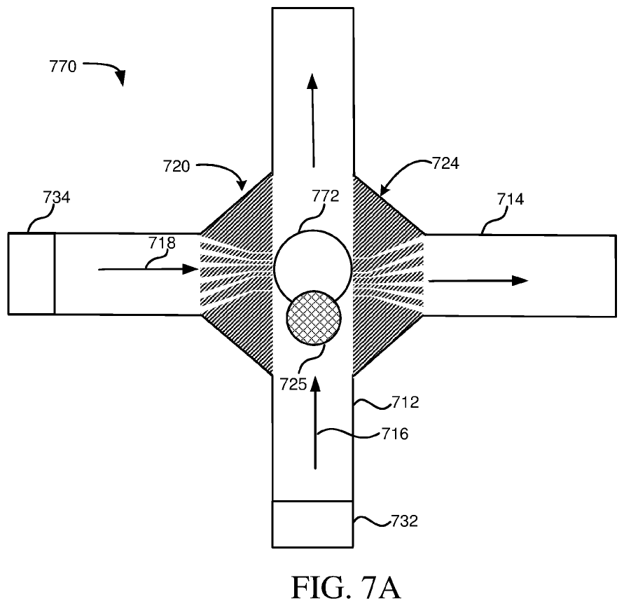

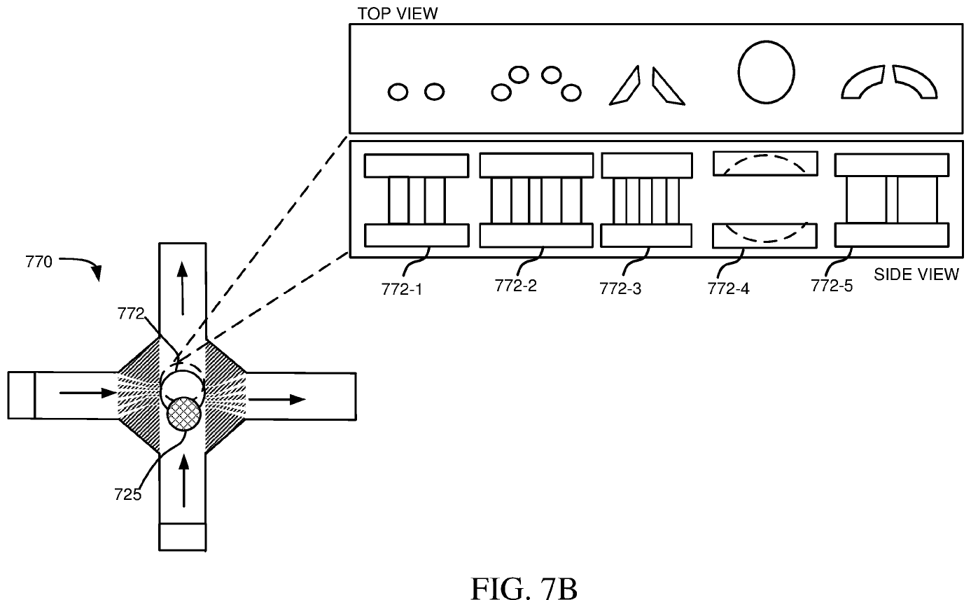

As previously described, apparatuses are not limited to channel walls formed by cylinder pillar structures spaced periodically. In various examples, as illustrated further by FIGS. 6-7, the channel walls may be formed by elongated pillar structures which may simplify the fabrication process as compared to the cylinder or other geometric shaped pillar structures which are not elongated and may additionally or alternatively reduce flow variant of interrogation and/or reduce impedance in the cross flow.

FIG. 6 illustrates another example apparatus for measuring deformability of a cell, consistent with examples of the present disclosure. The apparatus 660 of FIG. 6 is similar to the apparatus 330 of FIG. 3 but with the first channel wall 620, and the optional second channel wall 624 formed by a plurality of elongated pillars in the intersection of the first fluidic channel 612 and the second fluidic channel 614 of the microfluidic device. The apparatus 660 includes a microfluidic device having the first fluidic channel 612 coupled to a first fluidic pump 632, the second fluidic channel 614 coupled to a second fluidic pump 634, the first channel wall 620, and the optional second channel wall 624. Circuitry (not illustrated by FIG. 6) may be coupled to the first fluidic pump 632 and second fluidic pump 634 to control actuation of the fluidic pumps 632, 634 and to drive fluid in a first direction 616 and in a second direction 618.

The first channel wall 620 (and the optional second channel wall 624) may be formed by a plurality of elongated pillar structures, as illustrated by the labeled elongated pillar structure 662. The plurality of elongated pillar structures are spaced periodically in the intersection to form a plurality of apertures that provide fluidic communication between the first and second fluidic channels 612, 614. The plurality of elongated pillar structures modify the second pressure field generated by the second fluidic pump 634 and create a structured pressure field that is exerted on a cell 625 located in the intersection. The fluid flowing in the first direction 616 and in the second direction 618 may cross one another and result in the structured pressure field. To prevent or mitigate side shift of the cell 625 and/or stiction of the cell 625 to the second channel wall 624, the fluid flowing in the first direction 616 and in the second direction 618 may be controlled by the circuitry. For example, the direction of flow in the second fluidic channel 614 may be switched, such as from left-to-right (e.g., the second direction 618) and right-to-left (e.g., a third direction that is opposite of or 180 degrees from the second direction 618) of FIG. 6, every other pulse to avoid stiction while providing a side shift that averages to zero over an even number of pulses.

The apertures formed by the spacings between the elongated pillars may effectively form sub-channels for the second pressure field generated by the second fluidic pump 634 to flow through. The first fluidic channel 612 may be of a size that does not exceed the pitch of the elongated pillar structures for deformation interrogation. For example, for a first channel wall 620 exhibiting a width of apertures of 1-10 μm (and in some examples 1-5 μm), a height of elongated pillar structures of 20 um, and a pitch between the elongated pillar structures of 20 μm, the first fluidic channel 612 may have a width of 20 μm and a height of 20 μm, although examples are not so limited.

FIGS. 7A-7B illustrates an example apparatus for measuring deformability of a cell including a barrier to contain a cell, consistent with examples of the present disclosure. The apparatus 770 of FIG. 7A is similar to the apparatus 330 of FIG. 3 with the first channel wall 720, and the optional second channel wall 724 formed by a plurality of elongated pillars in the intersection of the first fluidic channel 712 and the second fluidic channel 714 of a microfluidic device. The apparatus 770 includes the first fluidic channel 712 coupled to a first fluidic pump 732, the second fluidic channel 714 coupled to a second fluidic pump 734, the first channel wall 720, and the optional second channel wall 724. The apparatus 770 may further include circuitry to control actuation of the fluidic pumps 732, 734 and to drive fluid in a first direction 716 and in a second direction 718, as previously described.

The first channel wall 720 (and the optional second channel wall 724) is formed by a plurality of elongated pillar structures, as noted above. The apertures of the first channel wall 720 are not limited to symmetrical geometric shapes. As shown, the plurality of elongated pillar structures are spaced periodically in the intersection to form a plurality of funnel-shaped apertures to provide fluidic communication between the first and second fluidic channels 712, 714. The plurality of elongated pillar structures modify the second pressure field generated by the second fluidic pump 734 and create the structured pressure field that is applied to a cell 725 located in the intersection.

To prevent or mitigate side shift of the cell 725 and/or stiction of the cell 725 to the second channel wall 724, the fluid flowing in the first direction 716 and in the second direction 718 may be controlled by the circuitry. Alternatively and/or in addition, the intersection may include a barrier 772 to contain the cell 725.

FIG. 7B illustrates different example barriers which may be used in a variety of apparatuses. As previously described, an apparatus 770 may include a barrier 772 in an intersection between two fluidic channels to contain the cell 725. Example barriers 772-1, 772-2, 772-3, 772-4 and 772-5 illustrate various designs of a barrier 772 that may be used. As illustrated, barrier 772-1 may include two pillars disposed orthogonal to the flow of the biologic sample to trap the cell 725 for measuring deformability.

As a further example, a pillar trap 772-2 may be disposed orthogonal to the flow of the biologic sample. Similar to the two pillars illustrated in 772-1, the pillar trap 772-2 may include a plurality of vertically aligned pillars to trap the cell 725 for measuring deformability.

In yet another example, a funnel 772-3 may be disposed orthogonal to the flow of the biologic sample. The funnel 772-3 may include two tapered members, vertically aligned orthogonal to the flow of the biologic sample. The tapered members may trap the cell 725 for measuring deformability.

Furthermore, a depression 772-4 may be disposed orthogonal to the flow of the biologic sample. The depression 772-4 may include a recessed portion of the substrate and lid of the microfluidic device. The depression 772-4 may trap the cell 725 for measuring deformability.

Yet further, a wall 772-5 may be disposed orthogonal to the flow of the biologic sample. The wall 772-5 may include a plurality of curved orthogonal pillars within the microfluidic device. The wall 772-5 may be disposed orthogonal to the flow of the biologic sample and may trap the cell 725 for measuring deformability.

Although barriers 772-1, 772-2, 772-3, 772-4 and 772-5 illustrate different kinds of structures that can facilitate the trapping of the cell 725 in the intersection, different and/or additional barriers may be used. In various examples, once measurements are obtained, the first fluidic pump may be actuated to move the cell 725 around the example barriers 772-1, 772-2, 772-3, 772-4 and 772-5. For example, the cell 725 may be released from the barrier 772 by reversing the flow momentarily, before re-establishing the flow in the direction illustrated. Similarly, the various example apparatuses illustrated herein, such as the apparatus 210 of FIG. 2, may include a barrier.

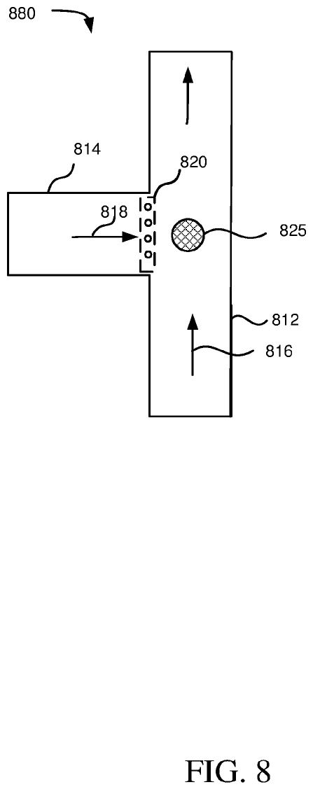

FIG. 8 illustrates another example apparatus for measuring deformability of a cell, consistent with examples of the present disclosure. As previously described, example apparatuses are not limited to cross-sectional fluidic channels and may include a T-flow array. In such examples, the apparatus 880 includes a first fluidic channel 812 and a second fluidic channel 814 that intersects the first fluidic channel 812 and that ends or terminates at the intersection. Fluid flows in a first direction 816 along the first fluidic channel 812 via a first fluidic pump and flows in a second direction 818 along the second fluidic channel 814 via a second fluidic pump. The intersection between the first fluidic channel 812 and the second fluidic channel 814 includes a channel wall 820 having a plurality of apertures that create the structured pressure field in response to fluid flowing therethrough, as previously described. The structured pressure field is exerted on the cell 825 and deformation of the cell is 825 observed.

The T-flow array may prevent or mitigate side shift of the cell 825 under deformation interrogation and may simplify apparatus control. The example apparatus 880 with the T-flow array may include a variety of a variations. For example, although the channel wall 820 is illustrated as a plurality of pillar structures periodically spaced, examples are not so limited and may include elongated pillar structures, solid wall structures with apertures formed therein, and/or structures which are aperiodically spaced. In further examples, the second fluidic channel 814 may be actuated by a plurality of fluidic pumps, such as a plurality of TIJ ejectors, among other examples. Additionally and/or alternatively, external fluidic pumps may be used.

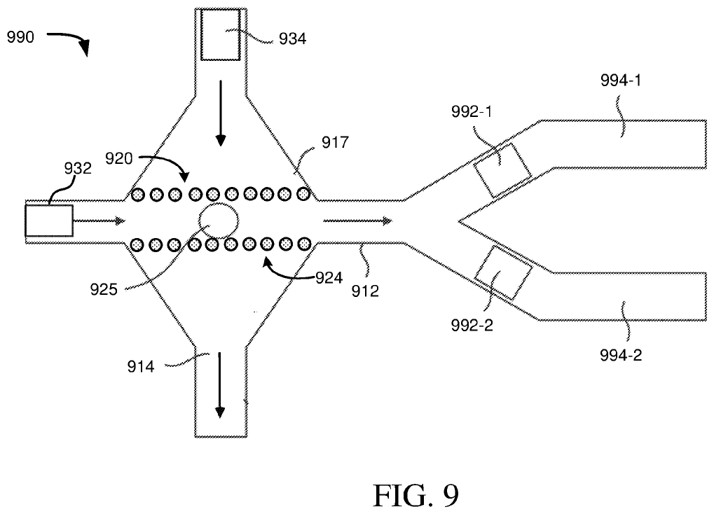

FIG. 9 illustrates another example apparatus for measuring deformability of a cell, consistent with the present disclosure. As illustrated in FIG. 9, the apparatus 990 may include a plurality of fluidic channels 912, 914, 994-1, 994-2 fluidically coupled to the cell probing chamber 917 to sort and concentrate cells after deformation analysis. The cell probing chamber 917 is located at the intersection of the first fluidic channel 912 and the second channel 914, and fluid is driven in two crossing directions (as illustrated by the arrows) by a first fluidic pump 932 and a second fluidic pump 934 of a set of fluidic pumps. As the properties of the cell 925 are determined via application of the structured pressure field created by the first channel wall 920 (and the optional second channel wall 924), one of a third and fourth fluidic pump 992-1, 992-2 (or more) of the set of fluidic pumps may fire to pull the cell 925 into the associated branching fluidic channel, 994-1, 994-2, respectively. The branching fluidic channels 994-1, 994-2 are coupled to the output of the first fluidic channel 912.

As an example, if deformability of the cell 925 is detected to be above a particular threshold, the cell 925 may be drawn into fluidic channel 994-1 by firing fluidic pump 992-2 to push the cell 925 into fluidic channel 994-1. Similarly, if deformability of the cell 925 is detected to be below the particular threshold, the cell 925 may be drawn into fluidic channel 994-2 by firing fluidic pump 992-1 to push the cell 925 into fluidic channel 994-2. Although FIG. 9 illustrates two branching fluidic channels 994-1, 994-2 fluidically coupled to the output of the first fluidic channel 912, examples are not so limited, and any number of fluidic channels may be coupled to the output of the first fluidic channel 912. Multiple channels may be of particular interest for cell sorting and concentration in different cell collectors.

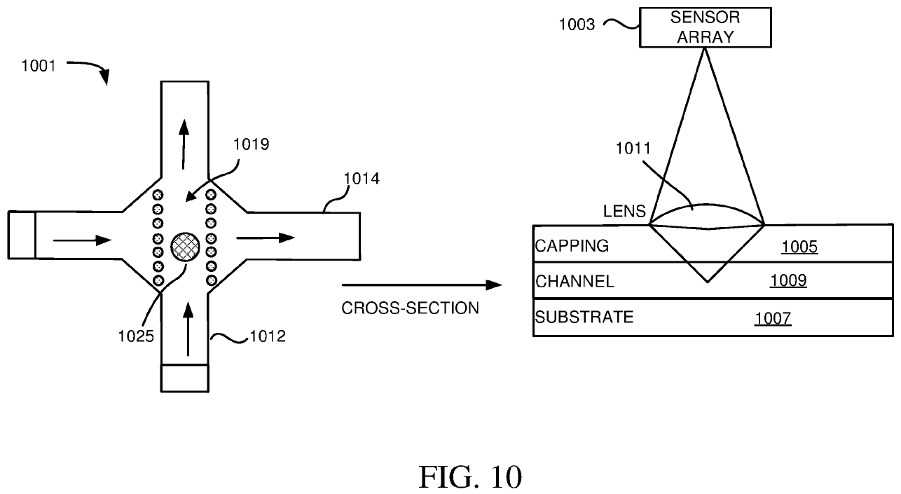

FIG. 10 illustrates another example apparatus for measuring deformability of a cell, consistent with the present disclosure. In particular, FIG. 10 illustrates an apparatus 1001 including an integrated optics system. As illustrated, the apparatus 1001 may include microfluidic device including a first fluidic channel 1012, a second fluidic channel 1014, and an intersection 1019 there between that forms a cell probing chamber. The cell probing chamber, as formed by the intersection 1019, may include a transparent lid 1005 disposed over a base substrate 1007 to form a channel 1009 therethrough. An integrated lens 1011 may be disposed on the transparent lid 1005 of the cell probing chamber. The integrated lens 1011 may focus light from the cell 1025 in the cell probing chamber to a sensor array 1003.

The integrated lens 1011 may comprise a plurality of structures. For instance, the integrated lens 1011 may comprise a zone plate, a Fresnel lens, metasurfaces, or other suitable lenses and/or micro-lenses for a variety of imaging modalities and optical configurations (e.g., infinity corrected, point-to-point magnification, integrated source, fluorescence, etc.). If a flat lens is used, the sensor may be in close proximity to the channel 1009 and substrate 1007 to create a compact package.

Circuitry as used herein, such as circuitry 336, 436, 536, may include a processor, machine readable instructions, and other electronics for communicating with and controlling the fluidic pumps, and other components of the apparatus, such as the imaging system, internal sensors, and other components. The circuitry may receive data from a host system, such as a computer, and includes memory for temporarily storing data. The data may be sent to the apparatus along an electronic, infrared, optical, or other information transfer path. A processor may be a central processing unit (CPU), a semiconductor-based microprocessor, a graphics processing unit (GPU), a microcontroller, special purpose logic hardware controlled by microcode or other hardware devices suitable for retrieval and/or execution of instructions stored in a memory, or combinations thereof. In addition to or alternatively to retrieving and executing instructions, the processor may include at least one integrated circuit (IC), other control logic, other electronic circuits, or combinations thereof that include a number of electronic components for performing the function. In some examples, the circuitry includes non-transitory computer-readable storage medium that is encoded with a series of executable instructions that may be executed by the processor. Non-transitory computer-readable storage medium may be an electronic, magnetic, optical, or other physical storage device that contains or stores executable instructions. Thus, non-transitory computer-readable storage medium may be, for example, Random Access Memory (RAM), an Electrically Erasable Programmable Read-Only Memory (EEPROM), a storage device, an optical disc, etc. In some examples, the computer-readable storage medium may be a non-transitory storage medium, where the term ‘non-transitory’ does not encompass transitory propagating signals.

A biologic sample, as used herein, generally refers to any biological material, collected from a subject. Examples of biologic samples include, but are not limited to, whole blood, blood plasma, and other body fluids, as well as tissue cell cultures obtained from humans, plants, or animals. Such biologic samples may contain any viral or cellular material, including all prokaryotic or eukaryotic cells, viruses, bacteriophages, mycoplasmas, protoplasts, and organelles. Such biological material may comprise all types of mammalian and non-mammalian animal cells, plant cells, algae including blue-green algae, fungi, bacteria, protozoa, etc. Non-limiting examples of samples include whole blood and blood-derived products such as plasma, serum and buffy coat, urine, feces, cerebrospinal fluid or any other body fluids, tissues, cell cultures, cell suspensions, etc.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein. Therefore, it is intended that this disclosure be limited only by the claims and the equivalents thereof.

1. A method, comprising:
flowing a biologic sample containing a plurality of cells along a first fluidic channel and into an intersection between the first fluidic channel and a second fluidic channel of a microfluidic device;
introducing a pressure field at the intersection and into the first fluidic channel via the second fluidic channel and a plurality of apertures in a channel wall disposed in the intersection; and
measuring deformability of a cell among the plurality of cells responsive to the introduction of the pressure field.

2. The method of claim 1, wherein introducing the pressure field includes generating a cross-directional flow to the flow of the biologic sample through the first fluidic channel.

3. The method of claim 1, wherein the intersection includes a cell probing chamber, and the method further comprises:
detecting the cell of the biologic sample in the cell probing chamber; and
isolating the cell in the intersection by reducing the flow of the biologic sample through the first fluidic channel.

4. The method of claim 1, further comprising imaging the cell while the pressure field causes deformation of the cell via an imaging system and measuring the deformability of the cell based on image data received from the imaging system.

5. The method of claim 1, wherein flowing the biologic sample includes flowing the biologic sample in a first direction along the first fluidic channel via a first fluidic pump, and reducing the flow in the first direction in response to the cell being located in the intersection, and introducing the pressure field in a second direction that crosses the first direction via a second fluidic pump.

6. The method of claim 1, further comprising sensing entry of the cell into the intersection via a signal received from a sensor disposed proximate to the intersection.

7. The method of claim 1, wherein introducing the pressure field includes driving a flow of fluid along the second fluidic channel and toward the channel wall via the pressure field, and segmenting the flow of the fluid into different pressures zones in the intersection via the plurality of apertures in the channel wall.

8. An apparatus, comprising:
a first fluidic channel;
a second fluidic channel that intersects the first fluidic channel, wherein each of the first fluidic channel and the second fluidic channel are actuated in crossing directions by a set of fluidic pumps; and
a first channel wall disposed at the intersection of the first fluidic channel and the second fluidic channel, the first channel wall including a plurality of apertures that provide fluidic communication between the first fluidic channel and the second fluidic channel and that create a structured pressure field in the intersection when fluid flows therethrough.

9. The apparatus of claim 8, wherein the apparatus comprises a microfluidic device and the microfluidic device further includes the set of fluidic pumps, the set of fluidic pumps including:
a first fluidic pump coupled to the first fluidic channel to generate a first pressure field and flow a biologic sample containing a plurality of cells along the first fluidic channel in a first direction of the crossing directions; and
a second fluidic pump coupled to the second fluidic channel to generate a second pressure field and flow the fluid along the second fluidic channel in a second direction of the crossing directions toward the intersection and through the plurality of apertures of the first channel wall responsive to a cell of the plurality of cells being located in the intersection, the plurality of apertures to modify the second pressure field to create the structured pressure field.

10. The apparatus of claim 8, wherein the first channel wall includes a plurality of pillar structures that are spaced periodically in the intersection to form the plurality of apertures.

11. The apparatus of claim 8, further including a second channel wall disposed at the intersection of the first fluidic channel, the second channel wall being opposite the first channel wall within the intersection.

12. An apparatus, comprising:
a microfluidic device including:
a first fluidic channel;
a second fluidic channel that intersects the first fluidic channel; and
a first channel wall disposed at the intersection of the first fluidic channel and the second fluidic channel, the first channel wall including a plurality of apertures that provide fluidic communication between the first fluidic channel and the second fluidic channel;
a first fluidic pump coupled to the first fluidic channel;
a second fluidic pump coupled to the second fluidic channel; and
circuitry arranged with the first fluidic pump and the second fluidic pump to:
activate the first fluidic pump to generate a first pressure field and to flow a biologic sample containing a plurality of cells, via the first pressure field, along the first fluidic channel in a first direction and toward the intersection of the first fluidic channel and the second fluidic channel;
activate the second fluidic pump to generate a second pressure field and to flow fluid, via the second pressure field, along the second fluidic channel in a second direction toward the intersection and through the first channel wall; and
measure deformability of a cell among the plurality of cells responsive to the introduction of the second pressure field.

13. The apparatus of claim 12, wherein the second fluidic channel is disposed orthogonal to the first fluidic channel, and the first direction and second direction cross one another.

14. The apparatus of claim 12, wherein the first fluidic channel, the second fluidic channel, and the intersection include a transparent lid disposed over a base substrate to form a cross-channel, and the apparatus further includes:
an imaging system arranged with the microfluidic device to capture image data of the intersection, and the circuitry is to measure the deformability of the cell based on the image data received from the imaging system.

15. The apparatus of claim 12, further including a third fluidic pump coupled with the second fluidic channel and a second channel wall disposed in the intersection that is opposite the first channel wall, wherein the circuitry is further arranged with the third fluidic pump to:
activate the third fluidic pump to generate a third pressure field and to flow fluid, via the third pressure field, along the second fluidic channel in a third direction and through the second channel wall, wherein the second pressure field and the third pressure field converge at the intersection, and the second direction and the third direction are cross directional to the flow of the biologic sample through the first fluidic channel in the first direction.
